(19) United States
(12) Patent Application Publication
(10) Pub. No.: US 2013/0040354 A1
(43) Pub. Date: Feb. 14, 2013

(54) Biogas Production Process With Enzymatic Pre-Treatment

(71) Applicant: Hans Sejr Olsen, Holte (DK)

(72) Inventor: Hans Sejr Olsen, Holte (DK)

(73) Assignee: NOVOZYMES A/S, Bagsvaerd (DK)

(21) Appl. No.: 13/521,463

(22) PCT Filed: Jan. 24, 2011
(86) PCT No.: PCT/EP11/50900

§ 371 Date: Aug. 21, 2012

(30) Foreign Application Priority Data
Jan. 29, 2010 (EP) 10152160.7
Jul. 8, 2010 (EP) 10168854.7
Aug. 12, 2010 (EP) 10172579.4

(51) Int. Cl. C12P 5/02

(52) U.S. Cl. 435/167

(57) ABSTRACT

A biogas production process with enzymatic pre-treatment, said process comprising the steps of providing a slurry comprising a lignocellulose-containing material, water and one or more enzyme; allowing the one or more enzyme to degrade the lignocellulose-containing material at a suitable temperature and pH; and adding the enzyme-degraded material to a biogas digester tank at a suitable rate and ratio to effectively convert the material to biogas in the digester.

### FIELD OF THE INVENTION

The present invention relates to biogas production processes with enzymatic pre-treatment, said processes comprising the steps of providing a slurry comprising a lignocellulose-containing material, water and one or more enzyme; allowing the one or more enzyme to degrade the lignocellulose-containing material at a suitable temperature and pH; and adding the enzyme-degraded material to a biogas digester tank at a suitable rate and ratio to effectively convert the material to biogas in the digester.

### BACKGROUND OF THE INVENTION

Most natural plant based material comprises a significant amount of lignocellulosic fibres that are undigestible or only slowly digestible in many biological systems. This has the consequence that for many biological processes converting plant based material a significant fraction of the treated material will not be digested or only digested in a low degree during the treatment.

For example in a usual biogas production plant biomass is fermented under anaerobic conditions to form biogas and a waste material consisting, to a large extent, of lignocellulosic fibers that are hardly digested at all.

Producing fermentation products, such as, ethanol, from lignocellulose is known in the art and generally includes pre-treating, hydrolyzing and fermenting the material. Lignocellulose-containing feed stock can be hydrolyzed to release fermentable sugars (WO 2010/000858).

The structure of lignocellulose is not directly accessible to enzymatic hydrolysis. Therefore, the lignocellulose is pre-treated in order to break the lignin seal and disrupt the crystalline structure of cellulose. This may cause solubilization and saccharification of the hemicellulose fraction. The cellulose fraction is then hydrolyzed enzymatically, e.g., by cellulolytic enzymes, which degrades the carbohydrate polymers into fermentable sugars.

Current processes for producing biogas from biomass are not yet optimized to achieve the full theoretic conversion to biogas, a fibrous lignocellulosic waste-material remains which is not converted at all.

### SUMMARY OF THE INVENTION

The invention relates to a biogas production process comprising at least one separate enzymatic pre-treatment step, where liquefaction, solubilisation and pre-saccharification is performed of biomass raw material, such as, straw, maize husklage, maize cobs, maize silage, solid waste from food processing of vegetables like potatoes, carrots, peas and beans, banana peel, orange peel, apple peels, bagasse from sugar cane, sugar beet pulp; but also stillage material from production of alcohol and wine as well as spent grain from production of beer, whisky and fuel ethanol as well as palm fronds, palm fruits, empty palm fruit bunches or palm residues.

During the enzymatic liquefaction the polysaccharides like starch, hemicelluloses, mannan and cellulose is solubilised and converted to mainly oligosaccharides. The protein is hydrolysed to mainly peptides. The cellulose is converted to cellodextrins.

From the pre-treatment tank(s) the liquefied material is fed to a biogas digester tank in a rate and ratio that fits with the conversion rate to gas. In the liquefaction system, pH is kept at same pH as in the digester tank.

Before or during the pre-treatment, a milling of the biomass may be done, preferably a wet grinding, optionally facilitated by addition of the enzymes according to the invention. Temperature and pH is adjusted to allow the enzymes to function.

This biomass can be prewashed with a base, such as, caustic, lime or soda.

Several advantages are provided by the process of the invention, including but not limited to:
Higher conversion rate in the biogas digester tank.
Higher productivity per unit of volume in the digester tank.
Lower investment in tank capacity.
Higher gas production per tank volume.
More efficient conversion of the lignocellulosic material at higher dry matter concentration.
Reduced amounts of unconverted material in the purge.
Higher dry matter content in the unconverted solids.
No need for post-converter or storage tank.
Easier dewatering of unconverted material.
Easier cleaning of the gas phase.

The process principle of the invention is illustrated in FIG. 1.

Accordingly, in a first aspect, the invention relates to a biogas production process with enzymatic pre-treatment, said process comprising the steps of:
(a) providing a slurry comprising a lignocellulose-containing material, water and one or more enzyme;
(b) allowing the one or more enzyme to degrade the lignocellulose-containing material at a suitable temperature and pH; and
(c) adding the enzyme-degraded material to a biogas digester tank at a suitable rate and ratio to effectively convert the material to biogas in the digester.

### BRIEF DESCRIPTION OF DRAWINGS

FIG. 1 shows a schematic outline of the biogas production process principle of the invention, including the enzymatic hydrolysis pre-treatment step(s).

FIG. 2 shows the reactor setup of Example 4.

FIG. 3 shows the accumulated production of methane from raw bagasse and treated bagasse as disclosed in Example 4.

### DETAILED DESCRIPTION OF THE INVENTION

In the first aspect the invention relates to biogas processes comprising an enzymatic pre-treatment step, wherein lignocellulose-containing materials are hydrolyzed and/or liquified/solubilized.

The inventors have found that subjecting the lignocellulose-containing material to one or more enzyme activities in a pre-treatment, the lignocellulose-containing material can be made more accessible to the biogas process.

### Lignocellulose-Containing Material

The term “lignocellulose-containing material” means material primarily consisting of cellulose, hemicellulose, and lignin. Lignocellulose-containing material is often referred to as “biomass”. Woody biomass is about 45-50% cellulose, 20-25% hemicellulose and 20-25% lignin. Herbaceous materials have lower cellulose, lower lignin and higher hemicellulose contents.

Cellulose is a linear beta 1->4 linked polymer of glucose. It is the principal component of all higher plant cell walls. In nature cellulose exists in crystalline and amorphous states. The thermodynamic stability of the beta 1->4 linkage and the capacity of cellulose to form internal hydrogen bonds gives it great structural strength. Cellulose is degraded to glucose through hydrolytic cleavage of the glycosidic bond.

Hemicellulose is a term used to refer to a wide variety of heteropolysaccharides found in association with cellulose and lignin in both woody and herbaceous plant species. The sugar composition varies with the plant species, but in angiosperms, the principal hemicellulosic sugar is xylose. Like cellulose, xylose occurs in the beta 1->4 linked backbone of the polymer. In gymnosperms, the principal component sugar is mannose. Arabinose is found as a side branch in some hemicelluloses.

Lignin is a phenylpropane polymer. Unlike cellulose and hemicellulose, lignin cannot be depolymerized by hydrolysis. Cleavage of the principal bonds in lignin require oxidation.

The lignocellulose-containing material may be any material containing lignocellulose. In a preferred embodiment the lignocellulose-containing material contains at least 30 wt.-%, preferably at least 50 wt.-%, more preferably at least 70 wt.-%, even more preferably at least 90 wt.-% lignocellulose. It is to be understood that the lignocellulose-containing material may also comprise other constituents such as proteinaceous material, starchy material, and sugars, such as fermentable sugars and/or un-fermentable sugars.

Lignocellulose-containing material is generally found, for example, in the stems, leaves, hulls, husks, and cobs of plants or leaves, branches, and wood of trees. Lignocellulose-containing material can also be, but is not limited to, herbaceous material, agricultural residues, forestry residues, municipal solid wastes, waste paper, and pulp and paper mill residues. It is to be understood that lignocellulose-containing material may be in the form of plant cell wall material containing lignin, cellulose and hemicellulose in a mixed matrix.

In a preferred embodiment the lignocellulose-containing material is corn fiber, rice straw, wheat bran, pine wood, wood chips, poplar, bagasse, sugar beet pulp, paper and pulp processing waste.

Other examples include corn stover, corn fiber, hardwood, such as poplar and birch, softwood, cereal straw, such as, wheat straw, switch grass, Miscanthus, rice hulls, ensilaged material like beets, fodder beets, corn silage, or mixtures thereof.

In a preferred embodiment of the first aspect of the invention, the content of lignocellulose-containing material in the slurry is adjusted by continuous or stepwise addition of lignocellulose-containing material to the slurry during step (b).

When there is an abundance of pectin in the material, demethylation of the pectin occurs naturally, which over time can result in a drop in pH to acidic conditions, as low as about pH 6. However, many of the enzyme activities suitable for pre-treatment of lignocellulosic biomass material are more effective at neutral to basic pH values. Therefore, it may be necessary to adjust pH up to alkaline values after some time, if pectinaceous substrates are comprised in the slurry. Accordingly, after a drop in pH-value to acidic conditions due to degradation of pectin in the substrate, pH is adjusted to neutral or basic conditions before cell wall degrading enzymes are added that are mainly active above pH 7. Suitable enzymes for substrates containing pectin are, e.g., pectate lyase (EC 4.2.2.2), an enzyme which degrades pectin by beta-elimination and consequently also lowers the viscosity or pectin methylesterase (EC 3.1.1.11) which hydrolyses pectin.

### Pre-Treatment

The lignocellulose-containing material may be pre-treated in any suitable way. The pre-treatment is carried out before or at the same time as the enzymatic hydrolysis. The goal of pre-treatment is to reduce the particle size, separate and/or release cellulose; hemicellulose and/or lignin and in this way increase the rate of hydrolysis. Pre-treatment processes such as wet-oxidation and alkaline pre-treatment targets lignin, while dilute acid and auto-hydrolysis targets hemicellulose. Steam explosion is an example of a pre-treatment that targets lignin.

The pre-treatment step may be a conventional pre-treatment step using techniques well known in the art. In a preferred embodiment pre-treatment takes place in a slurry of lignocellulose-containing material and water. The lignocellulose-containing material may during pre-treatment be present in an amount between 10-80 wt.-%, preferably between 20-70 wt.-%, especially between 30-60 wt.-%, such as around 50 wt-%.

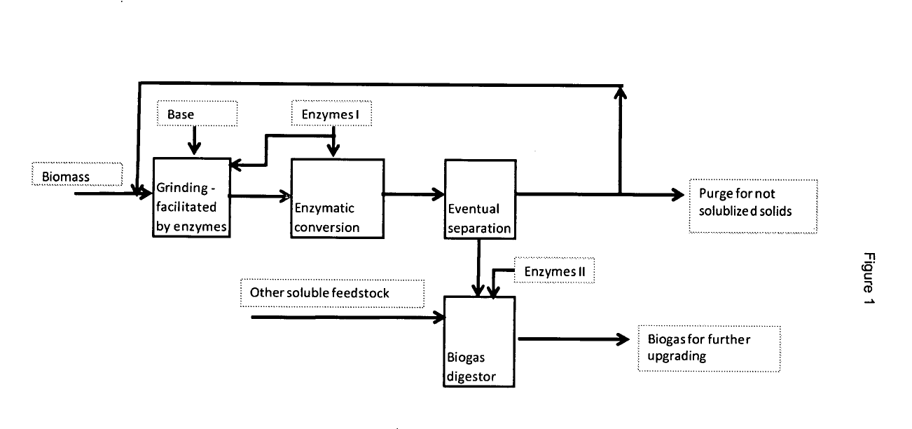

In a preferred embodiment of the first aspect of the invention, a solids separation step is performed after step (b) but before step (c) to purge not-solubilized solids (FIG. 1) and optionally feed them back into step (a) of the process.

Chemical, Mechanical and/or Biological Pre-Treatment

The lignocellulose-containing material may according to the invention be chemically, mechanically and/or biologically pre-treated before hydrolysis in accordance with the process of the invention. Mechanical pre-treatment (often referred to as “physical”-pre-treatment) may be carried out alone or may be combined with other pre-treatment processes.

Preferably, the chemical, mechanical and/or biological pre-treatment is carried out prior to the hydrolysis. Alternatively, the chemical, mechanical and/or biological pre-treatment may be carried out simultaneously with hydrolysis, such as simultaneously with addition of one or more hydrolyzing enzymes, and/or other enzyme activities, to release fermentable sugars, such as glucose and/or maltose.

### Chemical Pre-Treatment

The term “chemical pre-treatment” refers to any chemical pre-treatment which promotes the separation and/or release of cellulose, hemicellulose and/or lignin. Examples of suitable chemical pre-treatments include treatment with; for example, dilute acid, lime, alkaline, organic solvent, ammonia, sulfur dioxide, carbon dioxide. Further, wet oxidation and pH-controlled hydrothermolysis are also considered chemical pre-treatment.

Other pre-treatment techniques are also contemplated according to the invention. Cellulose solvent treatment has been shown to convert about 90% of cellulose to glucose. It has also been shown that enzymatic hydrolysis could be greatly enhanced when the lignocellulose structure is disrupted. Alkaline H₂O₂, ozone, organosols (uses Lewis acids, FeCl₃, Al₂(SO₄)₃ in aqueous alcohols), glycerol, dioxane, phenol, or ethylene glycol are among solvents known to disrupt cellulose structure and promote hydrolysis (Mosier et al. Bioresource Technology 96 (2005), p. 673-686).

Alkaline chemical pre-treatment with base, e.g., NaOH, Na₂CO₃, NaHCO₃, Ca(OH)₂, lime hydrate, ammonia and/or KOH or the like, is also within the scope of the invention. Pre-treatment processes using ammonia are described in, e.g., WO 2006/110891, WO 2006/11899, WO 2006/11900, WO 2006/110901, which are hereby incorporated by reference. Also the Kraft pulping process as described for example in “Pulp Processes” by Sven A. Rydholm, page 583-648. ISBN 0-89874-856-9 (1985) might be used. The solid pulp (about 50% by weight based on the dry wood chips) is collected and washed before the enzymatic treatments.

Wet oxidation techniques involve use of oxidizing agents, such as: sulphite based oxidizing agents or the like. Examples of solvent pre-treatments include treatment with DMSO (Dimethyl Sulfoxide) or the like. Chemical pre-treatment is generally carried out for 1 to 60 minutes, such as from 5 to 30 minutes, but may be carried out for shorter or longer periods of time dependent on the material to be pre-treated.

Other examples of suitable pre-treatment processes are described by Schell et al. (2003) Appl. Biochem and Biotechn. Vol. 105-108, p. 69-85, and Mosier et al. Bioresource Technology 96 (2005) 673-686, and US publication no. 2002/0164730, which references are hereby all incorporated by reference.

### Mechanical Pre-Treatment

The term “mechanical pre-treatment” refers to any mechanical (or physical) pre-treatment which promotes the separation and/or release of cellulose, hemicellulose and/or lignin from lignocellulose-containing material. For example, mechanical pre-treatment includes various types of milling, irradiation, steaming/steam explosion, and hydrothermolysis.

Mechanical pre-treatment includes comminution (mechanical reduction of the size). Comminution includes dry milling, wet milling and vibratory ball milling. Mechanical pre-treatment may involve high pressure and/or high temperature (steam explosion). In an embodiment of the invention high pressure means pressure in the range from 300 to 600 psi, preferably 400 to 500 psi, such as around 450 psi. In an embodiment of the invention high temperature means temperatures in the range from about 100 to 300° C., preferably from about 140 to 235° C. In a preferred embodiment mechanical pre-treatment is carried out as a batch-process, in a steam gun hydrolyzer system which uses high pressure and high temperature as defined above. A Sunds Hydrolyzer (available from Sunds Defibrator AB (Sweden) may be used for this.

In a preferred embodiment the lignocellulose-containing material is subjected to a irradiation pre-treatment. The term “irradiation pre-treatment” refers to any pre-treatment by microwave e.g. as described by Zhu et al. “Production of ethanol from microwave-assisted alkali pre-treated wheat straw” in Process Biochemistry 41 (2006) 869-873 or ultrasonic pre-treatment, e.g., as described by e.g. Li et al. “A kinetic study on enzymatic hydrolysis of a variety of pulps for its enhancement with continuous ultrasonic irradiation”, in Biochemical Engineering Journal 19 (2004) 155-164.

In another preferred embodiment, the lignocellulose-containing material or the slurry is homogenized; preferably by milling, wet-milling, grinding or wet-grinding prior to or during step (b).

### Combined Chemical and Mechanical Pre-Treatment

In a preferred embodiment the lignocellulose-containing material is subjected to both chemical and mechanical pre-treatment. For instance, the pre-treatment step may involve dilute or mild acid treatment and high temperature and/or pressure treatment. The chemical and mechanical pre-treatments may be carried out sequentially or simultaneously, as desired.

In a preferred embodiment the pre-treatment is carried out as a dilute and/or mild acid steam explosion step. In another preferred embodiment pre-treatment is carried out as an ammonia fiber explosion step (or AFEX pre-treatment step).

In yet another preferred embodiment, a base is added to the lignocellulose-containing material or the slurry prior to or while it is being homogenized; preferably the base is NaOH, Na₂CO₃, NaHCO₃, Ca(OH)₂, lime hydrate, ammonia and/or KOH.

### Biological Pre-Treatment

The term “biological pre-treatment” refers to any biological pre-treatment which promotes the separation and/or release of cellulose, hemicellulose, and/or lignin from the lignocellulose-containing material. Known biological pre-treatment techniques involve applying lignin-solubilizing microorganisms (see, for example, Hsu, T.-A., 1996, Pretreatment of biomass, in Handbook on Bioethanol: Production and Utilization, Wyman, C. E., ed., Taylor & Francis, Washington, D.C., 179-212; Ghosh, P., and Singh, A., 1993, Physicochemical and biological treatments for enzymatic/microbial conversion of lignocellulosic biomass, Adv. Appl. Microbial. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson, L., and Hahn-Hagerdal, B., 1996, Fermentation of lignocellulosic hydrolysates for ethanol production, Enz. Microb. Tech. 18: 312-331; and Vallander, L., and Eriksson, K.-E. L., 1990, Production of ethanol from lignocellulosic materials: State of the art, Adv. Biochem. Eng./Biotechnol. 42: 63-95).

### Enzymatic Hydrolysis

Before the pre-treated lignocellulose-containing material is fermented it is hydrolyzed enzymatically to break down especially hemicellulose and/or cellulose into fermentable sugars.

According to the invention the enzymatic hydrolysis is performed in several steps. The lignocellulose-containing material to be hydrolyzed constitutes above 2.5% wt-% DS (dry solids), preferably above 5% wt-% DS, preferably above 10% wt-% DS, preferably above 15 wt-% DS, preferably above 20 wt.-% DS, more preferably above 25 wt-% DS of the slurry of step a).

In step (b) of the invention, the lignocellulose-containing material is subjected to the action of one, or several or all enzyme activities selected from the group consisting of an amylolytic enzyme, a lipolytic enzyme, a proteolytic enzyme, a cellulolytic enzyme, an oxidoreductase and a plant cell-wall degrading enzyme.

In a preferred embodiment, the one or more enzyme is selected from the group consisting of aminopeptidase, alpha-amylase, amyloglucosidase, arabinofuranosidase, arabinoxylanase, beta-glucanase, carbohydrase, carboxypeptidase, catalase, cellobiohydrolase, cellulase, chitinase, cutinase, cyclodextrin glycosyltransferase, ferulic acid esterase, deoxyribonuclease, endo-cellulase, endo-glucanase, endo-xylanase, esterase, galactosidase, beta-galactosidase, glucoamylase, glucose oxidase, glucosidase, haloperoxidase, hemicellulase, invertase, isomerase, laccase, ligase, lipase, lyase, mannanase, mannosidase, oxidase, pectate lyase, pectin lyase, pectin trans-eliminase, pectin ethylesterase, pectin methylesterase, pectinolytic enzyme, peroxidase, protease, phytase, phenoloxidase, polygalacturonase, polyphenoloxidase, proteolytic enzyme, rhamnogalacturonan lyase, rhamnoglucanase, rhamnogalacturonase, ribonuclease, SPS-ase, transferase, transglutaminase, xylanase and xyloglucanase.

In another preferred embodiment, the one or more enzyme is a protease, a pectate lyase, a ferulic acid esterase and/or a mannanase.

It is noteworthy, that the pre-treated biomass material should preferably have a neutral to basic pH value when it is added to the biogas digester, it is thought that addition of acidic biomass may halt the biogas conversion process due to inhibition of the common methanogenic microorganisms.

In a preferred embodiment of the method of the first aspect, the pH is between 7 and 10, such as from 7.6 to 10; preferably from 8 to 10, or from 8 to 9, preferably around pH 8.5. The pH may be adjusted using NaOH, Na₂CO₃, NaHCO₃, Ca(OH)₂, lime hydrate, ammonia and/or KOH. The temperature may be between 20-70° C., preferably 30-60° C., and more preferably 40-55° C., e.g., around 50° C. During step (b) the cell walls are degraded and the cellulose fibrils are made accessible for further hydrolysis. The hydrolysis in step (b) may be carried out as a fed batch process where pre-treated lignocellulose-containing material is fed continuously/gradually or stepwise into a solution containing hydrolyzing enzymes.

In an embodiment a pectate lyase, a ferulic acid esterase, and a mannanase is present in the hydrolysis step (b). In an embodiment a pectate lyase, a ferulic acid esterase, mannanase and a cellulase is present. In an embodiment a pectate lyase, a ferulic acid esterase, mannanase, a cellulase and a protease is present.

Optionally, cellulose fibrils may be isolated and treated with an alkaline endo-glucanase composition under neutral to basic pH conditions. In that step, the dry solids (DS) is preferably above 10 wt.-% DS, preferably above 15 wt-% DS, preferably above 20 wt.-% DS, more preferably above 25 wt-% DS.

The pH should be between 7 and 10, such as from 8 to 9, preferably around pH 8.5. Prior to steps (a) or (b) the pH may be adjusted using NaOH, Na₂CO₃, NaHCO₃, Ca(OH)₂, lime hydrate, ammonia and/or KOH. The temperature may be between in range from 20-70° C., preferably 30-60° C., and more preferably 40-50° C.

The cellulose fibrils may be treated with a cellulase composition comprising cellulolytic activity under neutral to acid pH conditions. Preferably the pH is between 4-7, preferably 5-7, such as around 5.5. The pH is preferably adjusted using phosphoric acid, succinic acid, hydrochloric acid and/or sulphuric acid. Preferably with a temperature in the range of 20-70° C., preferably 30-60° C., and more preferably 40-50° C.

### Enzymes

Even if not specifically mentioned in context of a process or process of the invention, it is to be understood that the enzyme(s) (as well as other compounds) are used in an “effective amount”

### Proteases

Any protease suitable for use under alkaline conditions can be used. Suitable proteases include those of animal, vegetable or microbial origin. Microbial origin is preferred. Chemically or genetically modified mutants are included. The protease may be a serine protease, preferably an alkaline microbial protease or a trypsin-like protease. Examples of alkaline proteases are subtilisins, especially those derived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g. of porcine or bovine origin) and the Fusarium protease described in WO 89/06270.

Preferred commercially available protease enzymes include those sold under the trade names Everlase™, Kannase™, Alcalase™, Savinase™, Primase™, Durazym™, and Esperase™ by Novozymes A/S (Denmark), those sold under the tradename Maxatase, Maxacal, Maxapem, Properase, Purafect and Purafect OXP by Genencor International, and those sold under the tradename Opticlean and Optimase by Solvay Enzymes.

### Hemicellulolytic Enzymes

Any hemicellulase suitable for use in hydrolyzing hemicellulose, may be used. Preferred hemicellulases include pectate lyases, xylanases, arabinofuranosidases, acetyl xylan esterase, ferulic acid esterase, glucuronidases, endo-galactanase, mannases, endo or exo arabinases, exo-galactanses, and mixtures of two or more thereof. Preferably, the hemicellulase for use in the present invention is an endo-acting hemicellulase, and more preferably, the hemicellulase is an endo-acting hemicellulase which has the ability to hydrolyze hemicellulose under basic conditions of above pH 7, preferably pH 7-10.

In an embodiment the hemicellulase is a xylanase. In an embodiment the xylanase may preferably be of microbial origin, such as of fungal origin (e.g., Trichoderma, Meripilus, Humicola, Aspergillus, Fusarium) or from a bacterium (e.g., Bacillus). In a preferred embodiment the xylanase is derived from a filamentous fungus, preferably derived from a strain of Aspergillus, such as Aspergillus aculeatus; or a strain of Humicola, preferably Humicola lanuginosa. The xylanase may preferably be an endo-1,4-beta-xylanase, more preferably an endo-1,4-beta-xylanase of GH10 or GH11. Examples of commercial xylanases include SHEARZYME® 200L, SHEARZYME® 500L, BIOFEED WHEAT®, and PULPZYME™ HC (from Novozymes) and GC 880, SPEZYME® CP (from Genencor Int).

The hemicellulase may be added in an amount effective to hydrolyze hemicellulose, such as, in amounts from about 0.001 to 0.5 wt.-% of total solids (TS), more preferably from about 0.05 to 0.5 wt.-% of TS.

Xylanases may be added in the amounts of 1.0-1000 FXU/kg dry solids, preferably from 5-500 FXU/kg dry solids, preferably from 5-100 FXU/kg dry solids and most preferably from 10-100 FXU/kg dry solids.

Xylanases may alternatively be added in amounts of 0.001-1.0 g/kg DS substrate, preferably in the amounts of 0.005-0.5 g/kg DS substrate, and most preferably from 0.05-0.10 g/kg DS substrate.

### Pectolytic Enzymes (or Pectinases)

Any pectinolytic enzyme that can degrade the pectin composition of plant cell walls may be used in practicing the present invention. Suitable pectinases include, without limitation, those of fungal or bacterial origin. Chemically or genetically modified pectinases are also encompassed. Preferably, the pectinase used in the invention are recombinantly produced and are mono-component enzymes.

Pectinases can be classified according to their preferential substrate, highly methyl-esterified pectin or low methyl-esterified pectin and polygalacturonic acid (pectate), and their reaction mechanism, beta-elimination or hydrolysis. Pectinases can be mainly endo-acting, cutting the polymer at random sites within the chain to give a mixture of oligomers, or they may be exo-acting, attacking from one end of the polymer and producing monomers or dimers. Several pectinase activities acting on the smooth regions of pectin are included in the classification of enzymes provided by Enzyme Nomenclature (1992), e.g., pectate lyase (EC 4.2.2.2), pectin lyase (EC 4.2.2.10), polygalacturonase (EC 3.2.1.15), exo-polygalacturonase (EC 3.2.1.67), exo-polygalacturonate lyase (EC 4.2.2.9) and exo-poly-alpha-galacturonosidase (EC 3.2.1.82).

In embodiments the pectinase is a pectate lyase. Pectate lyase enzymatic activity as used herein refers to catalysis of the random cleavage of alpha-1,4-glycosidic linkages in pectic acid (also called polygalcturonic acid) by transelimination. Pectate lyases are also termed polygalacturonate lyases and poly(1,4-α-D-galacturonide) lyases.

The Pectate lyase (EC 4.2.2.2) is an enzyme which catalyse the random cleavage of α-1,4-glycosidic linkages in pectic acid (also called polygalacturonic acid) by transelimination. Pectate lyases also include polygalacturonate lyases and poly(1,4-α-D-galacturonide) lyases.

Examples of preferred pectate lyases are those that have been cloned from different bacterial genera such as Erwinia, Pseudomonas, Klebsiella, Xanthomonas and Bacillus, especially Bacillus licheniformis (U.S. Pat. No. 6,124,127), as well as from Bacillus subtilis (Nasser et al. (1993) FEBS Letts. 335:319-326) and Bacillus sp. YA-14 (Kim et al. (1994) Biosci. Biotech. Biochem. 58:947-949). Purification of pectate lyases with maximum activity in the pH range of 8-10 produced by Bacillus pumilus (Dave and Vaughn (1971) J. Bacteriol. 108:166-174), B. polymyxa (Nagel and Vaughn (1961) Arch. Biochem. Biophys. 93:344-352), B. stearothermophilus (Karbassi and Vaughn (1980) Can. J. Microbiol. 26:377-384), Bacillus sp. (Hasegawa and Nagel (1966) J. Food Sci. 31:838-845) and Bacillus sp. RK9 (Kelly and Fogarty (1978) Can. J. Microbiol. 24:1164-1172) have also been described.

A preferred pectate lyase may be obtained from Bacillus licheniformis as described in U.S. Pat. No. 6,124,127.

Other pectate lyases could be those that comprise the amino acid sequence of a pectate lyase disclosed in Heffron et al., (1995) Mol. Plant-Microbe Interact. 8: 331-334 and Henrissat et al., (1995) Plant Physiol. 107: 963-976.

A single enzyme or a combination of pectate lyases may be used. A preferred commercial pectate lyase preparation suitable for the invention is BioPrep® 3000 L available from Novozymes A/S.

### Mannanases

In the context of the present invention a mannanase is a beta-mannanase and defined as an enzyme belonging to EC 3.2.1.78.

Mannanases have been identified in several Bacillus organisms. For example, Talbot et al., Appl. Environ. Microbiol., Vol. 56, No. 11, pp. 3505-3510 (1990) describes a beta-mannanase derived from Bacillus stearothermophilus having an optimum pH of 5.5-7.5. Mendoza et al., World J. Microbiol. Biotech., Vol. 10, No. 5, pp. 551-555 (1994) describes a beta-mannanase derived from Bacillus subtilis having an optimum activity at pH 5.0 and 55° C. JP-03047076 discloses a beta-mannanase derived from Bacillus sp., having an optimum pH of 8-10. JP-63056289 describes the production of an alkaline, thermostable beta-mannanase. JP-08051975 discloses alkaline beta-mannanases from alkalophilic Bacillus sp. AM-001. A purified mannanase from Bacillus amyloliquefaciens is disclosed in WO 97/11164. WO 94/25576 discloses an enzyme from Aspergillus aculeatus, CBS 101.43, exhibiting mannanase activity and WO 93/24622 discloses a mannanase isolated from Trichoderma reesei.

The mannanase may be derived from a strain of the genus Bacillus, such as the amino acid sequence having the sequence deposited as GENESEQP accession number AAY54122 or an amino acid sequence which is homologous to this amino acid sequence. A suitable commercial mannanase preparation is Mannaway® produced by Novozymes A/S.

### Ferulic Esterases

In the context of the present invention a ferulic esterase is defined as an enzyme belonging to EC 3.1.1.73.

A suitable ferulic esterase preparation can be obtained from Malabrancea, e.g., from P. cinnamomea, such as e.g. a preparation comprising the ferulic esterase having the amino acid sequence shown in SEQ ID NO:2 in European patent application number 07121322.7, or an amino acid sequence which is homologous to this amino acid sequence.

Another suitable ferulic esterase preparation can be obtained from Penicillium, e.g., from P. aurantiogriseum, such as e.g. a preparation comprising the ferulic esterase having the amino acid sequence shown in SEQ ID NO:2 in European patent application number 0815469.7, or an amino acid sequence which is homologous to this amino acid sequence. A suitable commercial ferulic esterase preparation is NOVOZYM® 342 L produced by Novozymes A/S.

### Alkaline Endo-Glucanases

The term “endoglucanase” means an endo-1,4-(1,3;1,4)-beta-D-glucan 4-glucanohydrolase (E.C. No. 3.2.1.4), which catalyses endo-hydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. Alkaline endo-glucanases are endo-glucanases having activity under alkaline conditions.

In a preferred embodiment endoglucanases may be derived from a strain of the genus Trichoderma, preferably a strain of Trichoderma reesei; a strain of the genus Humicola, such as a strain of Humicola insolens; or a strain of Chrysosporium, preferably a strain of Chrysosporium lucknowense.

In a preferred embodiment endoglucanases may be derived from a strain of the genus Bacillus akibai.

In an embodiment the alkaline endo-glucanase composition is one of the commercially available products CAREZYME®, ENDOLASE® and CELLUCLEAN® (Novozymes A/S, Denmark). The enzyme may be applied in a dosage of 1-100 g/kg cellulose.

### Acid Cellulolytic Activity

The term “acid cellulolytic activity” as used herein are understood as comprising enzymes having cellobiohydrolase activity (EC 3.2.1.91), e.g., cellobiohydrolase I and/or cellobiohydrolase II, as well as endo-glucanase activity (EC 3.2.1.4) and/or beta-glucosidase activity (EC 3.2.1.21) having activity at pH below 6.

The cellulolytic activity may, in a preferred embodiment, be in the form of a preparation of enzymes of fungal origin, such as from a strain of the genus Trichoderma, preferably a strain of Trichoderma reesei; a strain of the genus Humicola, such as a strain of Humicola insolens; or a strain of Chrysosporium, preferably a strain of Chrysosporium lucknowense.

In preferred embodiment the cellulolytic enzyme preparation contains one or more of the following activities: endoglucanase, cellobiohydrolases I and II, and beta-glucosidase activity.

In a preferred embodiment cellulolytic enzyme preparation is a composition disclosed in WO2008/151079, which is hereby incorporated by reference. In a preferred embodiment the cellulolytic enzyme preparation comprising a polypeptide having cellulolytic enhancing activity, preferably a family GH61A polypeptide, preferably those disclosed in WO 2005/074656 (Novozymes). The cellulolytic enzyme preparation may further comprise beta-glucosidase, such as beta-glucosidase derived from a strain of the genus Trichoderma, Aspergillus or Penicillium, including the fusion protein having beta-glucosidase activity disclosed in co-pending application U.S. 60/832,511 (Novozymes). In a preferred embodiment the cellulolytic enzyme preparation may also comprises a CBH II enzyme, preferably Thielavia terrestris cellobiohydrolase II (CEL6A). In another preferred embodiment the cellulolytic enzyme preparation may also comprise cellulolytic enzymes; preferably those derived from Trichoderma reesei or Humicola insolens.

The cellulolytic enzyme composition may also comprising a polypeptide having cellulolytic enhancing activity (GH61A) disclosed in WO 2005/074656; a beta-glucosidase (e.g., fusion protein disclosed in U.S. 60/832,511 and PCT/US2007/074038), and cellulolytic enzymes derived from Trichoderma reesei. The cellulolytic enzyme composition.

In another preferred embodiment the cellulolytic composition comprising a polypeptide having cellulolytic enhancing activity (GH61A) disclosed in WO 2005/074656; a beta-glucosidase (e.g., fusion protein disclosed in U.S. 60/832,511 and PCT/US2007/074038), Thielavia terrestris cellobiohydrolase II (CEL6A), and cellulolytic enzymes preparation derived from Trichoderma reesei.

In an embodiment the cellulolytic enzyme composition is the commercially available product CELLUCLAST™ 1.5L, CELLUZYME™ (Novozymes A/S, Denmark) or ACCELLARASE™ 1000 (Genencor Int, Inc., USA).

The cellulolytic activity may be dosed in the range from 0.1-100 FPU per gram total solids (TS), preferably 0.5-50 FPU per gram TS, especially 1-20 FPU per gram TS.

### Cellulolytic Enhancing Activity

The term “cellulolytic enhancing activity” is defined herein as a biological activity that enhances the hydrolysis of a lignocellulose derived material by proteins having cellulolytic activity. For purposes of the present invention, cellulolytic enhancing activity is determined by measuring the increase in reducing sugars or in the increase of the total of cellobiose and glucose from the hydrolysis of a lignocellulose derived material, e.g., pre-treated lignocellulose-containing material by cellulolytic protein under the following conditions: 1-50 mg of total protein/g of cellulose in PCS (pre-treated corn stover), wherein total protein is comprised of 80-99.5% w/w cellulolytic protein/g of cellulose in PCS and 0.5-20% w/w protein of cellulolytic enhancing activity for 1-7 day at 50° C. compared to a control hydrolysis with equal total protein loading without cellulolytic enhancing activity (1-50 mg of cellulolytic protein/g of cellulose in PCS).

The polypeptides having cellulolytic enhancing activity enhance the hydrolysis of a lignocellulose derived material catalyzed by proteins having cellulolytic activity by reducing the amount of cellulolytic enzyme required to reach the same degree of hydrolysis preferably at least 0.1-fold, more at least 0.2-fold, more preferably at least 0.3-fold, more preferably at least 0.4-fold, more preferably at least 0.5-fold, more preferably at least 1-fold, more preferably at least 3-fold, more preferably at least 4-fold, more preferably at least 5-fold, more preferably at least 10-fold, more preferably at least 20-fold, even more preferably at least 30-fold, most preferably at least 50-fold, and even most preferably at least 100-fold.

In a preferred embodiment the hydrolysis and/or fermentation is carried out in the presence of a cellulolytic enzyme in combination with a polypeptide having enhancing activity. In a preferred embodiment the polypeptide having enhancing activity is a family GH61A polypeptide. WO 2005/074647 discloses isolated polypeptides having cellulolytic enhancing activity and polynucleotides thereof from Thielavia terrestris. WO 2005/074656 discloses an isolated polypeptide having cellulolytic enhancing activity and a polynucleotide thereof from Thermoascus aurantiacus. U.S. Published Application Serial No. 2007/0077630 discloses an isolated polypeptide having cellulolytic enhancing activity and a polynucleotide thereof from Trichoderma reesei.

### Alpha-Amylase

According to the invention any alpha-amylase may be used, such as of fungal, bacterial or plant origin. In a preferred embodiment the alpha-amylase is an acid alpha-amylase, e.g., acid fungal alpha-amylase or acid bacterial alpha-amylase. The term “acid alpha-amylase” means an alpha-amylase (E.C. 3.2.1.1) which added in an effective amount has activity optimum at a pH in the range of 3 to 7, preferably from 3.5 to 6, or more preferably from 4-5.

### Bacterial Alpha-Amylase

According to the invention a bacterial alpha-amylase is preferably derived from the genus Bacillus.

In a preferred embodiment the Bacillus alpha-amylase is derived from a strain of Bacillus licheniformis, Bacillus amyloliquefaciens, Bacillus subtilis or Bacillus stearothermophilus, but may also be derived from other Bacillus sp. Specific examples of contemplated alpha-amylases include the Bacillus licheniformis alpha-amylase shown in SEQ ID NO: 4 in WO 99/19467, the Bacillus amyloliquefaciens alpha-amylase SEQ ID NO: 5 in WO 99/19467 and the Bacillus stearothermophilus alpha-amylase shown in SEQ ID NO: 3 in WO 99/19467 (all sequences hereby incorporated by reference). In an embodiment the alpha-amylase may be an enzyme having a degree of identity of at least 60%, preferably at least 70%, more preferred at least 80%, even more preferred at least 90%, such as at least 95%, at least 96%, at least 97%, at least 98% or at least 99% to any of the sequences shown in SEQ ID NOS: 1, 2 or 3, respectively, in WO 99/19467.

The Bacillus alpha-amylase may also be a variant and/or hybrid, especially one described in any of WO 96/23873, WO 96/23874, WO 97/41213, WO 99/19467, WO 00/60059, and WO 02/10355 (all documents hereby incorporated by reference). Specifically contemplated alpha-amylase variants are disclosed in U.S. Pat. Nos. 6,093,562, 6,297,038 or U.S. Pat. No. 6,187,576 (hereby incorporated by reference) and include Bacillus stearothermophilus alpha-amylase (BSG alpha-amylase) variants having a deletion of one or two amino acid in positions R179 to G182, preferably a double deletion disclosed in WO 1996/023873—see e.g., page 20, lines 1-10 (hereby incorporated by reference), preferably corresponding to delta(181-182) compared to the wild-type BSG alpha-amylase amino acid sequence set forth in SEQ ID NO:3 disclosed in WO 99/19467 or deletion of amino acids R179 and G180 using SEQ ID NO:3 in WO 99/19467 for numbering (which reference is hereby incorporated by reference). Even more preferred are Bacillus alpha-amylases, especially Bacillus stearothermophilus alpha-amylase, which have a double deletion corresponding to delta(181-182) and further comprise a N193F substitution (also denoted I181*+G182*+N193F) compared to the wild-type BSG alpha-amylase amino acid sequence set forth in SEQ ID NO:3 disclosed in WO 99/19467.

In an embodiment the bacterial alpha-amylase is dosed in an amount of 0.0005-5 KNU per g DS, preferably 0.001-1 KNU per g DS, such as around 0.050 KNU per g DS.

### Fungal Alpha-Amylase

Fungal alpha-amylases include alpha-amylases derived from a strain of the genus Aspergillus, such as, Aspergillus oryzae, Aspergillus niger and Aspergillis kawachii alpha-amylases.

A preferred acidic fungal alpha-amylase is a Fungamyl-like alpha-amylase which is derived from a strain of Aspergillus oryzae. According to the present invention, the term “Fungamyl-like alpha-amylase” indicates an alpha-amylase which exhibits a high identity, i.e. at least 70%, at least 75%, at least 80%, at least 85%, at least 90%, at least 95%, at least 96%, at least 97%, at least 98%, at least 99% or even 100% identity to the mature part of the amino acid sequence shown in SEQ ID NO: 10 in WO 96/23874.

Another preferred acid alpha-amylase is derived from a strain Aspergillus niger. In a preferred embodiment the acid fungal alpha-amylase is the one from Aspergillus niger disclosed as “AMYA_ASPNG” in the Swiss-prot/TeEMBL database under the primary accession no. P56271 and described in WO 89/01969 (Example 3—incorporated by reference). A commercially available acid fungal alpha-amylase derived from Aspergillus niger is SP288 (available from Novozymes A/S, Denmark).

Other contemplated wild-type alpha-amylases include those derived from a strain of the genera Rhizomucor and Meripilus, preferably a strain of Rhizomucor pusillus (WO 2004/055178 incorporated by reference) or Meripilus giganteus.

In a preferred embodiment the alpha-amylase is derived from Aspergillus kawachii and disclosed by Kaneko et al. J. Ferment. Bioeng. 81:292-298 (1996) “Molecular-cloning and determination of the nucleotide-sequence of a gene encoding an acid-stable alpha-amylase from Aspergillus kawachii.”; and further as EMBL:#AB008370.

The fungal alpha-amylase may also be a wild-type enzyme comprising a starch-binding domain (SBD) and an alpha-amylase catalytic domain (i.e., none-hybrid), or a variant thereof. In an embodiment the wild-type alpha-amylase is derived from a strain of Aspergillus kawachii.

An acid alpha-amylases may according to the invention be added in an amount of 0.001 to 10 AFAU/g DS, preferably from 0.01 to 5 AFAU/g DS, especially 0.3 to 2 AFAU/g DS or 0.001 to 1 FAU-F/g DS, preferably 0.01 to 1 FAU-F/g DS.

### Commercial Alpha-Amylase Products

Preferred commercial compositions comprising alpha-amylase include MYCOLASE™ from DSM (Gist Brocades), BAN™, TERMAMYL™ SC, FUNGAMYL™, LIQUOZYME™ X, LIQUOZYMET™ SC and SAN™ SUPER, SAN™ EXTRA L (Novozymes A/S) and CLARASET™ L-40,000, DEX-LO™, SPEZYME™ FRED, SPEZYME™ AA, and SPEZYME™ DELTA AA (Genencor Int.), and the acid fungal alpha-amylase sold under the trade name SP288 (available from Novozymes A/S, Denmark).

### Carbohydrate-Source Generating Enzyme

The term “carbohydrate-source generating enzyme” includes glucoamylase (being glucose generators), beta-amylase and maltogenic amylase (being maltose generators) and also pullulanase and alpha-glucosidase. A carbohydrate-source generating enzyme is capable of producing a carbohydrate that can be used as an energy-source by the fermenting organism(s) in question, for instance, when used in a process of the invention for producing a fermentation product, such as ethanol. The generated carbohydrate may be converted directly or indirectly to the desired fermentation product, preferably ethanol. According to the invention a mixture of carbohydrate-source generating enzymes may be used. Especially contemplated mixtures are mixtures of at least a glucoamylase and an alpha-amylase, especially an acid amylase, even more preferred an acid fungal alpha-amylase. The ratio between acid fungal alpha-amylase activity (FAU-F) and glucoamylase activity (AGU) (i.e., FAU-F per AGU) may in an embodiment of the invention be between 0.1 and 100, in particular between 2 and 50, such as in the range from 10-40.

### Glucoamylase

A glucoamylase used according to the invention may be derived from any suitable source, e.g., derived from a microorganism or a plant. Preferred glucoamylases are of fungal or bacterial origin, selected from the group consisting of Aspergillus glucoamylases, in particular Aspergillus niger G1 or G2 glucoamylase (Boel et al. (1984), EMBO J. 3 (5), p. 1097-1102), or variants thereof, such as those disclosed in WO 92/00381, WO 00/04136 and WO 01/04273 (from Novozymes, Denmark); the A. awamori glucoamylase disclosed in WO 84/02921, Aspergillus oryzae glucoamylase (Agric. Biol. Chem. (1991), 55 (4), p. 941-949), or variants or fragments thereof. Other Aspergillus glucoamylase variants include variants with enhanced thermal stability: G137A and G139A (Chen et al. (1996), Prot. Eng. 9, 499-505); D257E and D293E/Q (Chen et al. (1995), Prot. Eng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Eng. 10, 1199-1204.

Other glucoamylases include Athelia rolfsii (previously denoted Corticium rolfsii) glucoamylase (see U.S. Pat. No. 4,727,026 and (Nagasaka, Y. et al. (1998) “Purification and properties of the raw-starch-degrading glucoamylases from Corticium rolfsii”, Appl Microbiol Biotechnol 50:323-330), Talaromyces glucoamylases, in particular derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215).

Bacterial glucoamylases contemplated include glucoamylases from the genus Clostridium, in particular C. thermoamylolyticum (EP 135,138), and C. thermohydrosulfuricum (WO 86/01831) and Trametes cingulate, Pachykytospora papyracea; and Leucopaxillus giganteus all disclosed in WO 2006/069289; or Peniophora rufomarginata disclosed in PCT/US2007/066618; or a mixture thereof. Also hybrid glucoamylase are contemplated according to the invention. Examples the hybrid glucoamylases disclosed in WO 2005/045018. Specific examples include the hybrid glucoamylase disclosed in Table 1 and 4 of Example 1 (which hybrids are hereby incorporated by reference).

Contemplated are also glucoamylases which exhibit a high identity to any of above mention glucoamylases, i.e., at least 70%, at least 75%, at least 80%, at least 85%, at least 90%, at least 95%, at least 96%, at least 97%, at least 98%, at least 99% or even 100% identity to the mature enzymes sequences mentioned above.

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U and AMG™ E (from Novozymes A/S); OPTIDEX™ 300 (from Genencor Int.); AMIGASE™ and AMIGASE™ PLUS (from DSM); G-ZYME™ G900, G-ZYME™ and G990 ZR (from Genencor Int.).

Glucoamylases may in an embodiment be added in an amount of 0.0001-20 AGU/g DS, preferably 0.001-10 AGU/g DS, especially between 0.01-5 AGU/g DS, such as 0.1-2 AGU/g DS.

### Biogas

The term “biogas” is according to the invention intended to mean the gas obtained in a conventional anaerobic fermentor, the primary digester. The main component of biogas is methane and the terms “biogas” and “methane” are in this application and claims used interchangeably.

### Primary Digester

The term “primary digester” is in this application and claims intended to mean the container wherein anaerobic fermentation takes place and biogas is produced.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will prevail.

Various references are cited herein, the disclosures of which are incorporated by reference in their entireties. The present invention is further described by the following examples which should not be construed as limiting the scope of the invention.

### EXAMPLES

### Materials & Methods

### Cellulase Activity Using Filter Paper Assay (FPU Assay)

### 1. Source of Process

1.1 The process is disclosed in a document entitled “Measurement of Cellulase Activities” by Adney, B. and Baker, J. 1996. Laboratory Analytical Procedure, LAP-006, National Renewable Energy Laboratory (NREL). It is based on the IUPAC process for measuring cellulase activity (Ghose, T. K., Measurement of Cellulse Activities, Pure & Appl. Chem. 59, pp. 257-268, 1987.

### 2. Procedure

2.1 The process is carried out as described by Adney and Baker, 1996, supra, except for the use of a 96 well plates to read the absorbance values after color development, as described below.

### 2.2 Enzyme Assay Tubes:

2.2.1 A rolled filter paper strip (#1 Whatman; 1×6 cm; 50 mg) is added to the bottom of a test tube (13×100 mm).
2.2.2 To the tube is added 1.0 mL of 0.05 M Na-citrate buffer (pH 4.80).
2.2.3 The tubes containing filter paper and buffer are incubated 5 min. at 50° C. (±0.1° C.) in a circulating water bath.
2.2.4 Following incubation, 0.5 mL of enzyme dilution in citrate buffer is added to the tube. Enzyme dilutions are designed to produce values slightly above and below the target value of 2.0 mg glucose.
2.2.5 The tube contents are mixed by gently vortexing for 3 seconds.
2.2.6 After vortexing, the tubes are incubated for 60 mins. at 50° C. (±0.1° C.) in a circulating water bath.
2.2.7 Immediately following the 60 min. incubation, the tubes are removed from the water bath, and 3.0 mL of DNS reagent is added to each tube to stop the reaction. The tubes are vortexed 3 seconds to mix.

### 2.3 Blank and Controls

2.3.1 A reagent blank is prepared by adding 1.5 mL of citrate buffer to a test tube.
2.3.2 A substrate control is prepared by placing a rolled filter paper strip into the bottom of a test tube, and adding 1.5 mL of citrate buffer.
2.3.3 Enzyme controls are prepared for each enzyme dilution by mixing 1.0 mL of citrate buffer with 0.5 mL of the appropriate enzyme dilution.
2.3.4 The reagent blank, substrate control, and enzyme controls are assayed in the same manner as the enzyme assay tubes, and done along with them.

### 2.4 Glucose Standards

2.4.1 A 100 mL stock solution of glucose (10.0 mg/mL) is prepared, and 5 mL aliquots are frozen. Prior to use, aliquots are thawed and vortexed to mix.
2.4.2 Dilutions of the stock solution are made in citrate buffer as follows:
   G1=1.0 mL stock+0.5 mL buffer=6.7 mg/mL=3.3 mg/0.5 mL
   G2=0.75 mL stock+0.75 mL buffer=5.0 mg/mL=2.5 mg/0.5 mL
   G3=0.5 mL stock+1.0 mL buffer=3.3 mg/mL=1.7 mg/0.5 mL
   G4=0.2 mL stock+0.8 mL buffer=2.0 mg/mL=1.0 mg/0.5 mL
2.4.3 Glucose standard tubes are prepared by adding 0.5 mL of each dilution to 1.0 mL of citrate buffer.
2.4.4 The glucose standard tubes are assayed in the same manner as the enzyme assay tubes, and done along with them.

### 2.5 Color Development

2.5.1 Following the 60 min. incubation and addition of DNS, the tubes are all boiled together for 5 mins. in a water bath.
2.5.2 After boiling, they are immediately cooled in an ice/water bath.
2.5.3 When cool, the tubes are briefly vortexed, and the pulp is allowed to settle. Then each tube is diluted by adding 50 microL from the tube to 200 microL of ddH2O in a 96-well plate. Each well is mixed, and the absorbance is read at 540 nm.
   2.6 Calculations (examples are given in the NREL document)
2.6.1 A glucose standard curve is prepared by graphing glucose concentration (mg/0.5 mL) for the four standards (G1-G4) vs. A540. This is fitted using a linear regression (Prism Software), and the equation for the line is used to determine the glucose produced for each of the enzyme assay tubes.
2.6.2 A plot of glucose produced (mg/0.5 mL) vs. total enzyme dilution is prepared, with the Y-axis (enzyme dilution) being on a log scale.
2.6.3 A line is drawn between the enzyme dilution that produced just above 2.0 mg glucose and the dilution that produced just below that. From this line, it is determined the enzyme dilution that would have produced exactly 2.0 mg of glucose.
2.6.4 The Filter Paper Units/mL (FPU/mL) are calculated as follows: FPU/mL=0.37/enzyme dilution producing 2.0 mg glucose

### Xylose/Glucose Isomerase Assay (IGIU)

1 IGIU is the amount of enzyme which converts glucose to fructose at an initial rate of 1 micromole per minute at standard analytical conditions.

Standard Conditions:

Glucose concentration: 45% w/w

pH: 7.5

Temperature: 60° C.

Mg²⁺ concentration: 99 mg/l (1.0 g/l MgSO₄*7H₂O)

Ca²⁺ concentration <2 ppm

Activator, SO₂ concentration: 100 ppm (0.18 g/l Na₂S₂O₅)

Buffer, Na₂CO₃, concentration: 2 mM Na₂CO₃

### Cellulytic Activity (EGU)

The cellulytic activity may be measured in endo-glucanase units (EGU), determined at pH 6.0 with carboxymethyl cellulose (CMC) as substrate.

A substrate solution is prepared, containing 34.0 g/l CMC (Hercules 7 LFD) in 0.1 M phosphate buffer at pH 6.0. The enzyme sample to be analyzed is dissolved in the same buffer. 5 ml substrate solution and 0.15 ml enzyme solution are mixed and transferred to a vibration viscosimeter (e.g. MIVI 3000 from Sofraser, France), thermostated at 40° C. for 30 minutes.

One EGU is defined as the amount of enzyme that reduces the viscosity to one half under these conditions. The amount of enzyme sample should be adjusted to provide 0.01-0.02 EGU/ml in the reaction mixture.

### Pectate Lyase Activity (APSU)

Pectate Lyase catalyses the formation of double bonds in polygalacturonic acid. The number of formed double bonds is determined by photometric measurement at 235 nm. One APSU (Alcalophile Pectate Lyase Unit) is defined as the amount of enzyme that produces C═C double bonds equivalent to 1 μmol unsaturated digalacturonic acid per minute under the standard conditions:

Temperature: 37.0° C.±0.5° C.

pH: 10.00±0.05

Wavelength: 235 nm in a 1 cm cuvette

Incubation time: 10 min.

Time of Measurement: 30 min.

Enzyme concentration range: 0.05-0.15 APSU/mL

Limit of quantification: 1.25 APSU/g

Range: [50; 150] mAPSU/mL

### Other Processes

Dry matter: Mettler Toledo HR 73 Halogen Moisture dryer

BRIX: RFM830 Digital refractometer from Bilingham & Stanley Ltd.

pH: WTW pH-meter

Milling: “coffee” grinder Bosch type KM13 (E nr: MKM 6003 FD 9512) for 2 minutes

HPLC: Waters 717 Autosampler, Waters 515 Pump and a Waters 2414 Refractive index detector. A column type Bio-rad (Animex HPX-87 H 300-7.8 mm), Cat no. 125140 was used. Standards were used for glucose, maltose, maltotriose, xylose, and maltotetraose

### Enzymes Used in the Examples:

A pectate lyase (EC 4.2.2.2) preparation derived from a Bacillus sp. and available from Novozymes as BioPrep® 3000 L with an activity of 3000 APSU/g composition.

An endo-xylanase (EC 3.2.1.8) composition derived from Bacillus agaradhaerens and available from Novozymes as Pulpzyme® HC.

Cellulase composition A comprising acid cellulolytic enzymes derived from Trichoderma reesei, a GH61A polypeptide disclosed in WO2005/074656, and an Aspergillus oryzae beta-glucosidase (in the fusion protein disclosed in WO2008/057637). Cellulase composition A is disclosed in WO2008/151079. Cellulase composition A has an activity 180 FPU/g composition.

Cellulase composition B comprising alkaline endo-cellulase derived from Bacillus sp. and available from Novozymes as Celluclean® Conc. with an activity of 320000 ECU/g composition.

Ferulic acid esterase composition also comprising alkaline cellulase. The composition is derived from Humicola insolens and available from Novozymes as Novozym® 342 with an activity of 90 EGU/g

Mannanase (EC 3.2.1.25) composition comprising a mannanase with an activity of 40 MIUM/g composition.

### Example 1

### Enzymatic Liquefaction of Biomass Raw Material for Biogas Production

A washing process under alkaline conditions was performed in order to remove soluble parts of the lignin and to swell the biomass material. The alkaline soluble compounds removed during the washing included unwanted inhibitor material for the microorganisms and the enzymes used during further processing. During washing or after the washing the biomass material was enzymatically liquefied using cell wall degrading enzymes and the recalcitrant structure of the biomass was opened so that the cellulose and other fermentable material could be easier digested.

The major structural polysaccharides of the lignocellulosic material in general consists of cellulose, hemicelluloses (rich in neutral sugars), pectin material containing D-galacturonic acid residues and mannan found in combination with lignin in various ratios in the cell walls of different plant species.
1. 200 g wheat straw material was milled using a coffee grinder, Bosch KM13 (E nr: MKM 6003 FD 9512) for 2 minutes. A slurry of the ground wheat straw was prepared using 2000 mL of 1.2% NaOH and slow stirring for 2 hours at room temperature.
2. The material was poured onto a screen sieve having a mesh size of 0.295 mm, it was washed on the screen using approximately 30 L of tap water and pressed using a spoon.
3. The dry matter content of the press cake was determined to be 9.44% w/w using a Mettler Toledo HR 73 Halogen Moisture dryer.
4. The pH of the pressed cake was measured to 8.3 using a WTW pH-meter.
5. 2000 g slurry having 6.7% w/w dry matter was prepared and divided into 4 reactors with 500 g in each, and the reactors were placed in a water bath at 50° C.
6. The enzyme dosages calculated per g of washed biomass shown in table 2 were used for the pre-treatment hydrolysis reaction in each reactor.
7. In order to verify by analyses that an enzymatic hydrolysis was going on samples were drawn at 0; 10; 60; 120 and 180 minutes.
8. The samples were analyzed for pH directly. After centrifuging a 10 mL sample for 10 minutes at 3800×G, the % solid phase was measured; results are shown in tables 3-5.
9. The supernatant was analyzed for % dry matter using a Mettler Toledo HR 73 Halogen Moisture dryer; results are shown in tables 3-5.
10. HPLC on the supernatant was run using a system consisting of a Waters 717 Autosampler, Waters 515 Pump and a Waters 2414 Refractive index detector. A column type Bio-rad (Animex HPX-87 H 300-7.8 mm; Cat no. 125140) was used. Standards were used for glucose, maltose, maltotriose, xylose, and maltotetraose. Note: Two as-of-yet unidentified tops were produced (work is ongoing). The results are shown in tables 4-5.

TABLE 1
Activities of the enzyme products used.

| | | | Novozym ® | |
|---|---|---|---|---|
| | BioPrep ® | Pulpzyme ® | 342 | |
| | 3000 L | HC | Cellulase & | Mannaway ® |
| Des- | Pectate | Endo- | Feruloyl | Conc. |
| cription | lyase | xylanase | esterase | Mannanase |
| Enzyme | 4.2.2.2 | 3.2.1.8 | 3.2.1.4 & | 3.2.1.25 |
| class (EC) | | | 3.1.1.73 | |
| Enzyme | 3000 APSU/g | 1000 AXU/g | 90 EGU/g | 40 MIUM/g |
| Activities | | | | |

TABLE 2
Activities used per g of biomass (dry matter) in the trials.

| Reactor | BioPrep ® | Pulpzyme ® | | Mannaway ® |
|---|---|---|---|---|
| no. | 3000 L | HC | Novozym ® 342 | Conc. |
| 1 | 15.00 APSU | 5.00 AXU | 0.45 EGU | 0.20 MIUM |
| 2 | — | 10.00 AXU | 0.45 EGU | 0.20 MIUM |
| 3 | — | 15.00 AXU | 0.45 EGU | — |
| 4 | — | 20.00 AXU | — | — |

TABLE 3
Direct measurements after 180 minutes reaction.

| | | | % dry matter of the |
|---|---|---|---|
| Reactor no. | pH | % Solid phase | supernatant |
| 1 | 8.2 | 20 | 2.0 |
| 2 | 8.2 | 25 | 1.1 |
| 3 | 8.2 | 18 | 1.1 |
| 4 | 8.3 | 18 | 0.8 |

TABLE 4
Measurements on reactor no. 1 versus time.

| | | | HPLC data: |
|---|---|---|---|
| Reaction | | | Σ of DP1 + DP2 + DP3 + |
| time | | Supernatants | C5 sugars + estimate for |
| (min.) | % solid phase | % dry matter | oligosaccharides + DP4, (g/L) |
| 0 | 55 | 0.20 | 6.2 |
| 10 | 15 | 0.20 | 3.3 |
| 60 | 20 | 0.53 | 4.9 |
| 120 | 25 | 1.05 | 7.5 |
| 180 | 20 | 1.98 | 14.8 |

TABLE 5
HPLC-data, g/L after 180 minutes reaction.

| | | | | Estimate of | | |
|---|---|---|---|---|---|---|
| | DP1 + | | C5 | oligo- | | Σ of all |
| Reactor | DP2 | DP3 | sugars | saccharides | DP4 | compounds |
| no. | (g/L) | (g/L) | (g/L) | (g/L) | (g/L) | (g/L) |
| 1 | 0.1 | 1.5 | 0.1 | 3.3 | 11.3 | 16.3 |
| 2 | 0.1 | 1.8 | 0.1 | 3.6 | 6.5 | 12.1 |
| 3 | 0.0 | 2.0 | 0.1 | 4.0 | 4.6 | 10.7 |
| 4 | 0.2 | 1.5 | 0.0 | 2.1 | 6.1 | 9.9 |

The hemicellulose hydrolysis of alkaline washed biomass (straw material) showed that BioPrep® (a pectolytic enzyme system) contained an important activity spectrum that enhanced the cell wall degrading effect in this alkaline region at pH=8-8.5. It was also shown that % dry matter of the supernatant and the sum of all compounds (g/L) correlated reasonably well.

Mainly DP4 was produced in significantly higher amount when BioPrep was included. When Novozym® 342 was included, the unidentified oligosaccharides were produced in higher amounts than when other enzyme systems were used. None of the enzyme systems used had significant saccharification effect on the cellulose microfibrils for glucose production revealing that they are mainly cell openers.

Values for the amounts of solubilized dry matter, high molecular compounds (DP4), and the sum of all DP's was in agreement with the high dry matter content found when the pectolytic enzyme system BioPrep® was applied in this pretreatment step.

### Example 2

### Enzymatic Digestion of Wheat Straw Biomass Material

1. 200 g wheat straw material was milled using a coffee grinder Bosch KM13 (E nr: MKM 6003 FD 9512) for 2 minutes. A slurry of the ground wheat straw was prepared using 2000 mL of 1.2% NaOH and slow stirring for 2 h at room temperature.
2. The material was poured onto a screen sieve having a mesh size of 0.295 mm, it was washed on the screen using approximately 15 L of tap water and pressed using a spoon.
3. The pH of the pressed cake was measured to 8.1 using a WTW pH-meter.
4. 2000 g slurry having 6.27% w/w dry matter was prepared and divided into 4 reactors with 500 g in each, and the reactors were placed in a water bath at 50° C.
5. The activities of the enzyme products used in the trial are shown in table 1 above.
6. The enzyme dosages calculated per g of washed biomass shown in table 6 were used for the pre-treatment.
7. A hydrolysis reaction was carried out in each reactor using the dosages shown in table 6.
8. In order to verify by analyses that an enzymatic hydrolysis was going on samples were drawn at 0; 10; 60; 120 and 180 minutes.
9. The samples were analyzed for pH directly. After centrifuging a 10 mL sample for 10 minutes at 3800×G, % solid phase was measured. Results are shown in tables 8-10.
10. The supernatant was analyzed for °Brix using a RFM830 Digital refractometer from Billingham and Stanley Ltd. Results are shown in tables 8-10.
11. HPLC on the supernatant was run using a system consisting of a Waters 717 Autosampler, Waters 515 Pump and a Waters 2414 Refractive index detector. A column type Bio-rad (Animex HPX-87 H 300-7.8 mm; cat. no. 125140) was used. Standards were used for glucose, maltose, maltotriose, xylose, and maltotetraose.

Results are shown in tables 8-10.

TABLE 6
Activities used per g of biomass (dry matter) in the trials.

| | BioPrep ® | Pulpzyme ® | Novozym ® | Mannaway ® |
|---|---|---|---|---|
| Reactor no. | 3000 L | HC | 342 | Conc. |
| 5 | 15.00 | — | 0.45 EGU | 0.20 MIUM |
| | APSU | | | |
| 6 | 15.00 | — | 0.45 EGU | — |
| | APSU | | | |
| 7 | 15.00 | 5.00 AXU | — | — |
| | APSU | | | |
| 8 | 15.00 | 5.00 AXU | — | 0.20 MIUM |
| | APSU | | | |

TABLE 8
Direct measurements after 180 minutes reaction.

| Reactor | | | °BRIX (estimate for % dry |
|---|---|---|---|
| no. | pH | % Solid phase | matter of the supernatant) |
| 5 | 8.1 | 20 | 0.9 |
| 6 | 8.1 | 14 | 0.8 |
| 7 | 8.2 | 25 | 0.8 |
| 8 | 8.1 | 18 | 0.7 |

TABLE 9
Measurements on reactor no. 5 versus time.

| Reaction | % | | HPLC data: Σ of DP1 + DP2 + DP3 + |
|---|---|---|---|
| time | solid | Supernatants | C5 sugars + estimate for |
| (minutes) | phase | °BRIX | oligosaccharides + DP4, (g/L) |
| 0 | 22 | 0.13 | 0.1 |
| 10 | 20 | 0.25 | 0.9 |
| 60 | 20 | 0.61 | 3.1 |
| 120 | 20 | 0.79 | 4.6 |
| 180 | 20 | 0.86 | 5.4 |

TABLE 10
HPLC-data, g/L after 180 minutes reaction.

| | | | | Estimate of | | |
|---|---|---|---|---|---|---|
| | DP1 + | | C5 | oligo- | | Σ of all |
| Reactor | DP2 | DP3 | sugars | saccharides | DP4 | compounds |
| no. | (g/L) | (g/L) | (g/L) | (g/L) | (g/L) | (g/L) |
| 5 | 0.1 | 1.2 | 0.07 | 1.5 | 2.5 | 5.4 |
| 6 | 0.1 | 1.1 | 0.05 | 1.5 | 1.9 | 4.7 |
| 7 | 0.2 | 0.8 | 0.01 | 0.9 | 3.4 | 5.3 |
| 8 | 0.2 | 0.8 | 0.07 | 0.8 | 3.0 | 4.8 |

With BioPrep® in the same dosage for hydrolysis of hemicellulose on alkaline washed biomass (straw material) a high solubilizing effect was seen in all 4 trials. Novozym® 342 solubilized slightly more carbohydrates than Pulpzyme. However, Pulpzyme HC released slightly more glucose and DP4.

### Example 3

### Enzymatic Digestion of Wheat Straw Biomass Material

1. 200 g wheat straw material was milled using a coffee grinder Bosch KM13 (E nr: MKM 6003 FD 9512) for 2 minutes. A slurry of the ground wheat straw was prepared using 2000 mL of 1.2% NaOH and slow stirring for 2 h at room temperature.
2. The material was poured onto a screen sieve having a mesh size of 0.295 mm, it was washed on the screen using approximately 30 L of tap water and pressed using a spoon.
3. The pH of the pressed cake was measured to 7.8 using a WTW pH-meter.
4. 2000 g slurry having 6.26% w/w dry matter was prepared and divided into 4 reactors with 500 g in each, and the reactors were placed in a water bath at 50° C.
5. The activities of the enzyme products used in the trial are shown in table 1 above.
6. The enzyme dosages calculated per g of washed biomass is shown in table 11 were used for the hydrolysis pre-treatment in each reactor.
7. In order to verify by analyses that an enzymatic hydrolysis is going on, samples were drawn at 0; 10; 60; 120 and 180 minutes. Results are shown in tables 12-14.
8. The samples were analyzed for pH directly. After centrifuging a 10 mL sample for 10 minutes at 3800×G, % solid phase was measured. Results are shown in tables 12-14.
9. The supernatant was analyzed for °Brix using a RFM830 Digital refractometer from Billingham and Stanley Ltd. Results are shown in tables 12-14.
10. HPLC on the supernatant was run using a system consisting of a Waters 717 Autosampler, Waters 515 Pump and a Waters 2414 Refractive index detector. A column type Bio-rad (Animex HPX-87 H 300-7.8 mm), Cat no. 125140 was used. Standards were used for glucose, maltose, maltotriose, xylose, and maltotetraose. Results are shown in tables 12-14.

TABLE 11
Activities used per g of biomass (dry matter) in the trials.

| Reactor | BioPrep ® | Novozym ® | Mannaway ® |
|---|---|---|---|
| no. | 3000 L | 342 | Conc. |
| 9 | 30.00 | 0.45 EGU | 0.20 MIUM |
| | APSU | | |
| 10 | 45.00 | 0.45 EGU | — |
| | APSU | | |
| 11 | 60.00 | — | — |
| | APSU | | |

TABLE 12
Direct measurements after 180 minutes reaction.

| | | | °BRIX (estimate for |
|---|---|---|---|
| | | | % dry matter of the |
| Reactor no. | pH | % Solid phase | supernatant) |
| 9 | 8.2 | 16 | 0.8 |
| 10 | 8.1 | 10 | 0.9 |
| 11 | 8.1 | 15 | 0.3 |

TABLE 13
Measurements on reactor no. 10 versus time.

| Reaction | % | | HPLC data: Σ of DP1 + DP2 + DP3 + |
|---|---|---|---|
| time | solid | Supernatants | C5 sugars + unidentified |
| (minutes) | phase | °BRIX | oligosaccharides + DP4, (g/L) |
| 0 | 45 | 0.1 | 0.2 |
| 10 | 25 | 0.4 | 1.7 |
| 60 | 15 | 0.6 | 3.5 |
| 120 | 15 | 0.8 | 4.7 |
| 180 | 10 | 0.9 | 5.5 |

TABLE 14
HPLC-data, g/L after 180 minutes reaction.

| | | | | Estimate of | | |
|---|---|---|---|---|---|---|
| | | | | unidentified | | |
| | DP1 + | | C5 | oligo- | | Σ of all |
| Reactor | DP2 | DP3 | sugars | saccharides | DP4 | compounds, |
| no. | (g/L) | (g/L) | (g/L) | (g/L) | (g/L) | (g/L) |
| 9 | 0.1 | 1.0 | 0.05 | 1.4 | 2.6 | 5.1 |
| 10 | 0.1 | 1.1 | 0.05 | 1.6 | 2.7 | 5.5 |
| 11 | 0.2 | 0.0 | 0.00 | 0.3 | 0.1 | 0.5 |

Generally, from the three examples, it can be concluded that the enzyme combination used in reactor no. 1 seems to be the optimal dosage so far, as shown in table 15 below. BioPrep® had no significant effect without any other cell wall degrading activities present. Pulpzyme had a favourable effect, especially on release of DP4.

TABLE 15
Best results re. alkaline cell wall opening.

| | % Dry | | | | |
|---|---|---|---|---|---|
| | matter | | | °BRIX (estimate | |
| | during | | | for % dry | Σ of all |
| Reactor | the | | % Solid | matter of the | compounds, |
| no. | reaction | pH | phase | supernatant) | (g/L) |
| 1 | 6.70 | 8.2 | 20 | 2.3 | 14.8 |
| 5 | 6.27 | 8.1 | 20 | 0.9 | 5.4 |

### Example 4

### Enzymatic Liquefaction and Digestion of Bagasse from Sugar Cane

Lignocellulosic material was washed under alkaline conditions to remove soluble compounds of the lignin and to swell the remaining material. The soluble compounds removed during the washing include enzyme inhibitors and material that inhibits growth of the microorganisms in the biogas digester. After the washing process the biomass material was wet milled and enzymatic liquefied using cell wall degrading enzymes. The recalcitrant structure of the biomass was opened so that cellulose, hemicelluloses and other fermentable material can be easier hydrolyzed and digested to biogas.

### Process Carried Out in Pilot Plant:

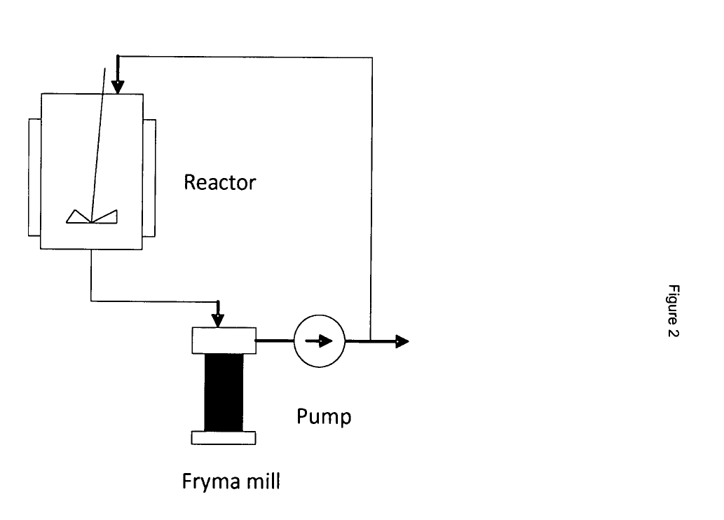

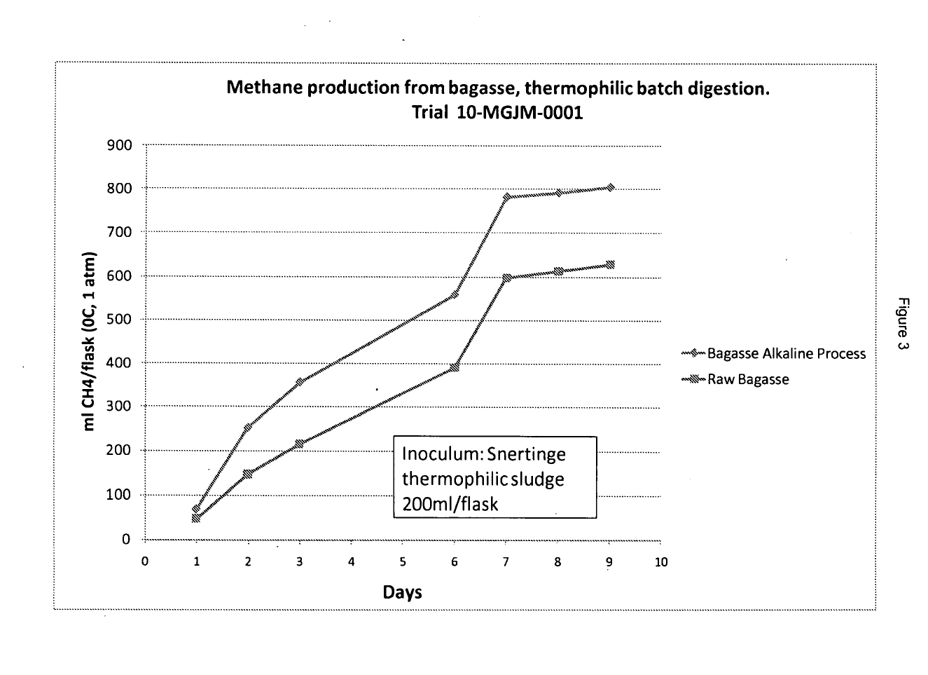

1. 5 kg raw bagasse consisting of pieces 1-2 cm was suspended in 22.5 litre of city water at 50° C. in a stirred container.
2. 0.6 kg 50% NaOH was added. This resulted in a concentration of 1.2% NaOH.
3. The alkaline treatment was performed during a gentle stirring for 2 hours at 50° C.
4. A wet screening was performed using an Algaier VTS 600 vibrating tumbler screen with a 40 p mesh screen. The solid phase was collected.
5. The solid phase was washed using 100 L city water (40-50° C.) and re-screened.
6. This procedure was repeated until pH was about 8.5 and most of the colour was removed.
7. The washed pulp was added water to a total volume 100 L and suspended by stirring. The material was pumped through a toothed colloid mill (Fryma Mill type MZ 110 adjusted to an opening of 1 mm) in a recirculation process once. This operation lasted about 30 minutes.
8. Hereafter the mash was treated by cell wall degrading enzyme activities, ferulic esterase, xylanase, pectate lyase, pectin lyase and endocellulase. Actually 25 g BioPrep® 3000 L, 25 g Novozym® 342, 25 g Pulpzyme® HC and 25 g Celluclean®5.0 L was added.
9. The enzyme process was carried out by pumping the reactor mixture through the Fryma mill using consecutive recirculations in time intervals of 60 minutes over a total period of 4 hours. The reactor setup is shown in FIG. 2. The Fryma mill was adjusted to a have 1 mm between rotor and stator.
10. A biogas trial was carried out in thermophilic biogas batch reactors of 200 mL using inoculums from a commercial waste treatment plant (Snertinge, Denmark). In the reactor a 1.67 g of dry matter of substrate was used. Methane production was measured once a day using a gas chromatograph. The accumulated productions of methane over 9 days are shown in FIG. 3.

We concluded that the alkaline enzymatic pre-treatment gave increased methane production when evaluated in the thermophilic batch digester system and when compared to the use of raw bagasse.

### Example 5

### Enzymatic Liquefaction and Digestion of Pelleted Wheat Straw (Fuel Pills)

### Process Carried Out in Pilot Plant:

1. 2.5 kg pelleted wheat straw (fuel pills) was suspended in 22.5 litre of city water at 40-50° C.
2. 1.85 kg 27% NaOH was added.
3. The alkaline treatment was performed during a gentle stirring for 2 hours at 50° C. pH was measured and a pH-value=12.3 was found.
4. A wet screening was performed using an Algaier VTS 600 vibrating tumbler screen with a 40 micron mesh screen. The solid phase was collected.
5. The solid phase was washed using 100 L water (40-50° C.) and re-screened. This procedure was repeated until pH was about 8.5 and most of the colour was removed. The results shown in table 16 were found.
6. The washed pulp was slurred in city water to a mass of 27 kg. It was heated to 45° C. and ph-adjusted from pH=8.7 to pH=8.0 using 17 mL 4 N HCl.
7. The slurry was treated on a toothed colloid mill (Fryma Mill type MZ 110, adjusted to an opening of 0.5 mm) for 40 minutes in a recirculation process and treated by cell wall degrading enzyme activities.
8. Hereafter the mash was treated by use of cell wall degrading enzyme activities, ferulic esterase, xylanase, pectate lyase, pectin lyase and endocellulase. Actually 25 g BioPrep® 3000 L, 25 g Novozym® 342, 25 g Pulpzyme® HC and 25 g Celluclean®5.0 L was added.
9. A consecutive recirculation of the reaction mixture through the Fryma mill was carried out in time intervals of 30 minutes over a total period of 6.5 hours. During the first 30 minutes an opening of the split between rotor and stator was 0.4 mm, during second period the split was 0.35 mm and during the third period the spilt was 0.30 mm. The data shown in table 17 were found.

TABLE 16
Screening results.

| Screening (no. | | | | % solids (10 mL |
|---|---|---|---|---|
| 2; no. 3; no. 4 | | | | sample centrifuged |
| washing with | | | | at 3000 × gravity for |
| 100 L city water) | kg fibre | kg filtrate | pH | 5 minutes) |
| 1 | 18.2 | 14.6 | 12.2 | 10 |
| 2 | 16.3 | 109 | 11.6 | <4 |
| 3 | 16.0 | 110 | 10.1 | <1 |
| 4 | 15.4 | 110 | 8.5 | <0.5 |

TABLE 17
Reaction results during the milling and reaction.

| | | | °Brix | % Solids (10 mL |
|---|---|---|---|---|
| | | | (refractometer | sample centrifuged |
| Reaction time | Temp | | dry matter | at 3000 × gravity for |
| (minutes) | (° C.) | pH | estimate) | 5 minutes) |
| 0 | 46.0 | 8.0 | 0.6 | 45 |
| 30 | 46.0 | 8.1-7.9 | 0.9 | 46 |
| 115 | 45.0 | 7.9 | — | — |
| 145 | 48.0 | 7.9 | 1.2 | 42 |
| 205 | 45.0 | 8.0 | — | — |
| 235 | 48.3 | 8.1 | 1.3 | 42 |
| 390 | 46.0 | 8.1-8.0 | 1.3 | 40 |
| 1350 (over | 45.0 | 7.1 | 1.6 | 39 |
| night) | | | | |

The fourth milling was carried next morning using a spilt of 0.20 mm. The viscosity was judged much lower than after 390 minutes. This was a clear indication that a significant liquefaction was obtained.

We concluded that the alkaline enzymatic pre-treatment process of the wheat straw material clearly reduced viscosity and opened the structure of the material. Thus it is expected that an increased methane production will be obtained when evaluated in a thermophilic batch biogas digester system.

### Example 6

### Production of Biogas from Sugar Beet Pulp

A pre-testing of our alkaline enzyme system consisting of NOVOZYM® 342, PULPZYME® HC, CELLUCLEAN® 5.0 L and BIOPREP® 3000 L (All from Novozymes A/S, Denmark) was performed on pre-milled samples supplied from Nordic Sugar, Nakskov, Denmark, as follows:
1. 10 g of sugar beet material was suspended in 20 g water at 50° C.
2. pH was adjusted to 8 using 4 N NaOH.
3. To the time t=0, 0.05 g (50 μL) of each of the enzyme products NOVOZYM® 342, PULPZYME® HC, CELLUCLEAN® 5.0 L and BIOPREP® 3000 L was added to the mix.
4. The reaction was carried out in a conical flask kept under stirring in a shaking table at 50° C.
5. After 10 minutes a Zero-sample was taken and frozen down for later assay. The samples are 2 mL. Samples are again taken at t=30 minutes, 60 minutes, 120 minutes and 240 minutes.
6. The assay was as follows; results are shown below in table 18:
   a. Centrifugation for 10 minutes at 14,000 RPM
   b. Measuring of degree °Brix.
   c. Measuring of absorbance at 235 nm in a quarts cuvette.

TABLE 18
Reaction results during the hydrolysis reaction of milled sugar beet
material.

| Time | Absorbance | | | |
|---|---|---|---|---|
| (min) | (235 nm) | Δ A(235) | °Brix | Δ Brix |
| 0 | 1.535 | 0 | 1.61 | 0 |
| 30 | 1.535 | 0 | 1.75 | 0.14 |
| 60 | 1.685 | 0.15 | 1.82 | 0.21 |
| 120 | 2.042 | 0.507 | 1.96 | 0.35 |
| 240 | 2.366 | 0.831 | 1.96 | 0.35 |
| 1080 | 3.35 | 1.815 | 2.44 | 0.83 |

Significant improved biogas production was found in a test system developed by Nordic Sugar and University of Hohenheim when compared to not pretreated sugar beet pulp (not shown).

### Example 7

### Enzymatic Hydrolysis of Pre-Milled Sugar Beet Pulp

This example illustrates enzymatic production of hydrolysate based on pre-milled sugar beet pulp supplied from Nordic Sugar, Nakskov, Denmark, as follows:
1. The dry matter content of the beet pulp was measured using the HR 73 Halogen moisture analyzer to: 15.01% w/w.
2. 150 g of beet pulp was blended by hand into 300 mL of city water in each of two flasks.
3. pH was measured and adjusted to approx. pH=8.5. Approximately 1.5 mL 4 N NaOH was added to each flask. Stirring was with a powerful stirrer used at 150 rpm. No enzymes were added to flask no. 1.
4. Enzymes were added to flask no. 2. A dosage of 0.25% enzyme product of dry matter was used of each of the 4 enzyme products mentioned above (and used in the examples 4, 5 and 6). The dry matter content of the reaction mixtures was estimated based on the masses and the measurement of the pulps dry matter content measured to 5.0%.
   Mass of dry matter: 150×15.01/100=22.5 (g dry matter used for dosage). This corresponded to 56.3 mg˜56.3/1.10˜50 μL, which was added.
5. pH and °Brix was measured and the reactions were continued overnight. The measurements are shown in Table 19.

TABLE 19
pH and Brix data.

| | | | °BRIX (of |
|---|---|---|---|
| | | | supernatant or |
| Date and time | Sample | pH | filtrate) |
| 01-06-2010 at 17:00 | Flask 1 | 6.35 (before | n.a. |
| | | adjustment). | |
| 01-06-2010 at 17:00 | Flask 2 | 6.25 (before | n.a |
| | | adjustment). | |
| 01-06-2010 at 17:45 | Flask 1 | 1.5 mL 4N NaOH | n.a. |
| | | was added: pH = 8.85 | |
| 01-06-2010 at 17:45 | Flask 2 | 1.5 mL 4N NaOH | n.a |
| | | was added 1.5 mL 4N | |
| | | NaOH: | |
| | | pH8.40 | |
| 01-06-2010 at 18:00 | Flask 1 | 8.30 | 1.49 |
| 01-06-2010 at 18:00 | Flask 2 | 7.90 | 1.28 |
| 02-06-2010 at 9:40 | Flask 1 | 5.14 | 1.62 |
| 02-06-2010 at 9:40 | Flask 2 | 5.15 | 1.28 |
| 02-06-2010 at 14:40 | Flask 1 | 5.07 | 1.62 |
| 02-06-2010 at 14:40 | Flask 2 | 5.35 | 1.35 |
| 02-06-2010 at 17:00 | Flask 1 | 4.72 | 1.35 (filtered) |
| 02-06-2010 at 17:00 | Flask 2 | 5.94 | 1.21 (filtered) |

A pH drop was detected for both flasks, probably due to demethylation. Methanol was detected in the reaction mixture after the enzymatic reaction (Flask no. 2) as shown by the HPLC result in table 20 below. The release of methanol as found in flask no. 2 might have a favourable influence of biogas production.

TABLE 20
HPLC-results after the alkaline enzymatic treatment

| | g/L | | | | | |
|---|---|---|---|---|---|---|
| | | | | Acetic | Lactic | % w/w |
| Sample | Glucose | Xylose | Arabinose | acid | acid | Methanol |
| Flask | 0.89 | 0.97 | 0 | 0.13 | 0.82 | 0.00 |
| no. 1 | | | | | | |
| (Blind) | | | | | | |
| Flask | | | 0 | | 0.36 | 0.42-0.47 |
| no. 2 | | | | | | |

1-14. (canceled)

15. A biogas production process with enzymatic pre-treatment, said process comprising the steps of:
(a) degrading a lignocellulose-containing material by one or more enzymes at a suitable temperature and pH in a slurry comprising the lignocellulose-containing material, water and the one or more enzymes; and
(b) adding the enzyme-degraded material to a biogas digester tank at a suitable rate and ratio to effectively convert the material to biogas in the digester.

16. The process of claim 15, wherein the one or more enzymes are selected from the group consisting of an amylolytic enzyme, a lipolytic enzyme, a proteolytic enzyme, a cellulolytic enzyme, an oxidoreductase and a plant cell-wall degrading enzyme.

17. The process of claim 16, wherein the one or more enzymes are selected from the group consisting of aminopeptidase, alpha-amylase, amyloglucosidase, arabinofuranosidase, arabinoxylanase, beta-glucanase, carbohydrase, carboxypeptidase, catalase, cellobiohydrolase, cellulase, chitinase, cutinase, cyclodextrin glycosyltransferase, ferulic acid esterase, deoxyribonuclease, endo-cellulase, endo-glucanase, endo-xylanase, esterase, galactosidase, beta-galactosidase, glucoamylase, glucose oxidase, glucosidase, haloperoxidase, hemicellulase, invertase, isomerase, laccase, ligase, lipase, lyase, mannanase, mannosidase, oxidase, pectate lyase, pectin lyase, pectin trans-eliminase, pectin ethylesterase, pectin methylesterase, pectinolytic enzyme, peroxidase, protease, phytase, phenoloxidase, polygalacturonase, polyphenoloxidase, proteolytic enzyme, rhamnogalacturonan lyase, rhamnoglucanase, rhamnogalacturonase, ribonuclease, SPS-ase, transferase, transglutaminase, xylanase and xyloglucanase.

18. The process of claim 16, wherein the one or more enzymes are a protease, a pectate lyase, a ferulic acid esterase and/or a mannanase.

19. The process of claim 15, further comprising homogenizing the lignocellulose-containing material or the slurry prior to or during step (a).

20. The process of claim 19, further comprising adding a base to the lignocellulose-containing material or the slurry prior to or while it is being homogenized.

21. The process of claim 20, wherein the base is NaOH, Na₂CO₃, NaHCO₃, Ca(OH)₂, lime hydrate, ammonia and/or KOH.

22. The process of claim 15, further comprising adjusting the content of the lignocellulose-containing material in the slurry by continuous or stepwise addition of the lignocellulose-containing material during preparation of the slurry.

23. The process of claim 15, wherein the lignocellulose-containing material constitutes above 2.5% wt. % dry solids (DS) of the slurry.

24. The process of claim 15, wherein step (a) is carried out at a pH in the range from 7 to 10.

25. The process of claim 15, wherein step (a) is carried out at a temperature in the range from 20-70° C.

26. The process of claim 15, further comprising a solids separation step after step (a) but before step (b) to purge not-solubilized solids and optionally feed them back into preparation of the slurry.

27. The process of claim 15, further comprising subjecting the lignocellulose-containing material to a microwave and/or an ultrasonic irradiation treatment prior to preparation of the slurry.

28. The process of claim 15, further comprising chemically, mechanical and/or biologically treating the lignocellulose-containing material prior to preparation of the slurry.

29. The process of claim 15, wherein the lignocellulose-containing material is derived from corn stover, corn fiber, hard wood, softwood, cereal straw, wheat straw, switch grass, Miscanthus, rice hulls, municipal solid waste, industrial organic waste, office paper, bagasse of sugar cane, sugar beet pulp, palm fronds, palm fruits, empty palm fruit bunches, palm residues or mixtures thereof.
